(19) United States
(12) Patent Application Publication
(10) Pub. No.: US 2016/0144143 A1
(43) Pub. Date: May 26, 2016

(54) ELECTRIC MEDICAMENT VAPORIZER

(71) Applicant: Martin Richard CARLSON, St. Thomas, VI (US)

(72) Inventor: Martin Richard CARLSON, St. Thomas, VI (US)

(21) Appl. No.: 14/865,517

(22) Filed: Sep. 25, 2015

Related U.S. Application Data
(60) Provisional application No. 62/083,457, filed on Nov. 24, 2014.

(51) Int. Cl. A61M 15/06; A61M 16/00; A61M 16/08; A61M 16/10

(52) U.S. Cl. CPC A61M 15/06; A61M 16/1095; A61M 16/0003; A61M 16/0875; A61M 2205/33; A61M 2205/3606

(57) ABSTRACT

An electric hookah for the delivery of medicaments having vaporizable active ingredients, wherein the hookah delivers the vapors at a relatively low temperature, and wherein the hookah is made of parts readily available at local hardware stores is disclosed. The upper tube may retain a heating assembly for heating a medicament so as to produce a vapor. The tubular base may also house a cooling assembly. The electric vaporizer may be adapted so that the vapor flows from the heating assembly, then through at least a portion of a screen assembly and then through the cooling assembly and to an outflow tube for an end user.

### CROSS-REFERENCE TO RELATED APPLICATION

This application claims the benefit of priority of U.S. provisional application No. 62/083,457, filed 24 Nov. 2014, the contents of which are herein incorporated by reference.

### BACKGROUND OF THE INVENTION

The present invention relates to vapor delivery systems and, more particularly, an electric hookah for the delivery of medicaments having vaporizable active ingredients, wherein the hookah delivers the vapors at a relatively low temperature.

Current waterless electric hookahs—i.e., hookahs where the user is not required to add water to the device prior to operation—provide two vessels, and so two hoses to hold at the same time, and as a result are inconvenient to use as they require two hands to properly manipulate each hose.

Current electric hookahs also typically do not provide temperature information related to their vaporizer chamber. Relatedly, said devices typically vaporize the subject medicament at relatively high temperatures, and so tend to produce harsh vapors.

Furthermore, current electric hookahs are not easily repairable or serviceable by the owner because their replacement parts are not easily found.

As can be seen, there is a need for an electric hookah for the delivery of medicaments having vaporizable active ingredients, wherein the hookah delivers the vapors at a relatively low temperature, and wherein the hookah is made of parts readily available at local hardware stores.

### SUMMARY OF THE INVENTION

In one aspect of the present invention, an electric hookah includes a tubular base forming a cavity for receiving a cooling fluid; an upper tube supported by the tubular base so that the only fluid communication with the cavity is via a condenser tube hole and an outflow tube hole; a heating element housed in the upper tube; a vapor tube having a thermal input and at least one egress, wherein the heating element is coupled to the thermal input; a flexible condenser tube fluidly interconnecting a first egress of the at least one egress and the condenser tube hole; and an outflow tube extending from the cavity and through the outflow tube hole.

In another aspect of the present invention, the electric hookah includes a tubular base forming a cavity for receiving a cooling fluid; an upper tube supported by the tubular base so that the only fluid communication with the cavity is via a condenser tube hole and an outflow tube hole; a support sleeve dispose between the tubular base and the upper tube, wherein an upper portion of the support sleeve slidably receives a lower portion of the upper tube, wherein the support sleeve forms the condenser tube hole and the outflow tube hole, wherein the lower portion of the upper tube forms a pair tube notches; a pair of aligned notches formed by the pairs of bowl and tube notches, wherein the pair of aligned notches receive a portion of the flexible condenser tube and the outflow tube, so that less than ten percent of the flexible condenser tube protrudes in the upper tube or the support sleeve; a heating element housed in the upper tube; a vapor tube having a thermal input and a first and a second egress, wherein the heating element is coupled to the thermal input; a flexible condenser tube fluidly interconnecting the first egress and the condenser tube hole; a thermal sensor operatively coupled to the second egress; an outflow tube extending from the cavity and through the outflow tube hole; two elbow tubes, a first elbow tube interconnecting the condenser tube hole and the condenser tube and a second elbow tube interconnecting the outflow tube hole and the outflow tube; a bowl tube interconnecting the cavity and the first elbow tube; and a cooling fluid in the cavity so that a portion of the bowl tube is submerged therein.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention;

FIG. 2 is an exploded view of an exemplary embodiment of the present invention;

FIG. 3 is a section view of an exemplary embodiment of the present invention, taken along line 3-3 in FIG. 1, with an soldering iron 32 and thermal sensor 24 simplified for clarity;

FIG. 4 is a detail section view of an exemplary embodiment of the present invention, taken along line 4-4 in FIG. 3;

FIG. 5 is a detail section view of an exemplary embodiment of the present invention;

FIG. 6 is a detail perspective view of an exemplary embodiment of the present invention with an elbow vapor tube 92 in place of a tee vapor tube 10;

FIG. 7 is a detail exploded view of an exemplary embodiment of the present invention with the elbow vapor tube 92; and

FIG. 8 is a section view of an exemplary embodiment of the present invention, taken along line 8-8 in FIG. 6.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides an electric vaporizer having a tubular base for supporting an upper tube in a vertical orientation. The upper tube may retain a heating assembly for heating a medicament so as to produce a vapor therefrom. The tubular base may also house a cooling assembly. The electric vaporizer may be adapted so that the vapor flows from the heating assembly, through at least a portion of a screen assembly, then bypassing the upper tube via flexible condenser tube, and then through the cooling assembly and to an outflow tube for an end user.

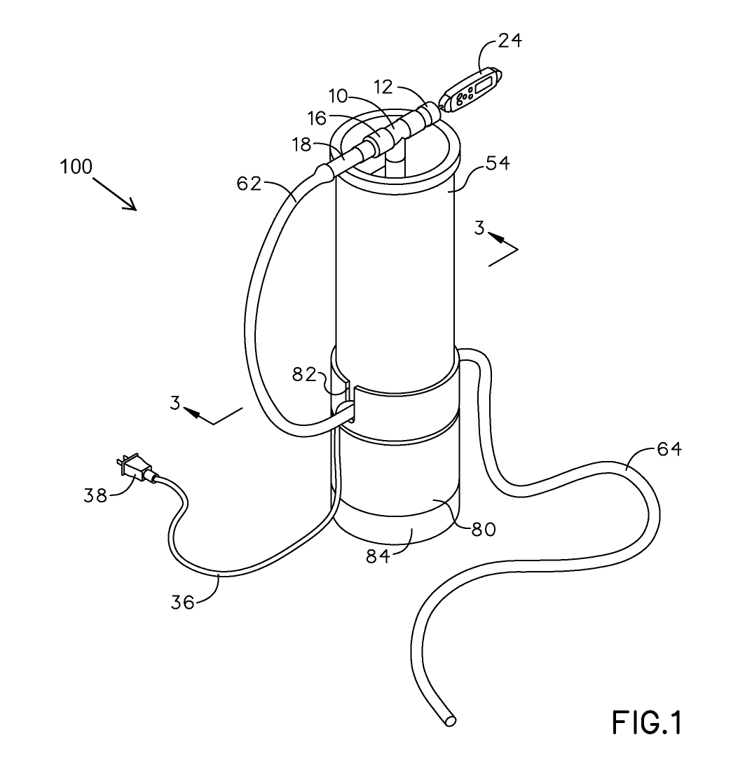

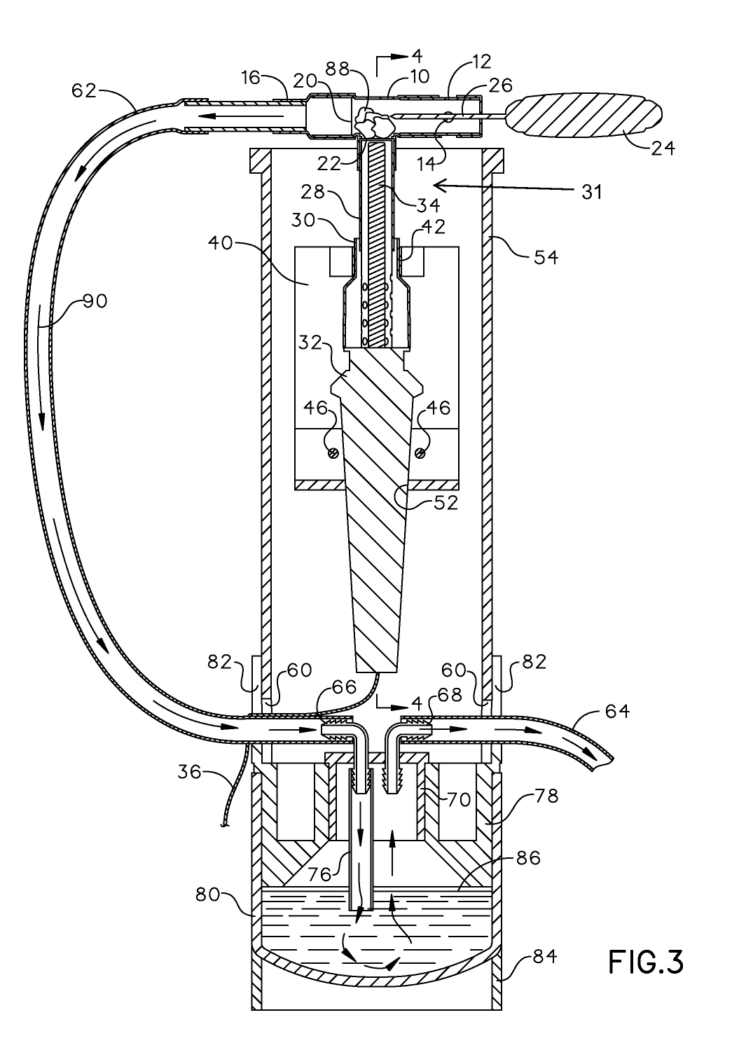

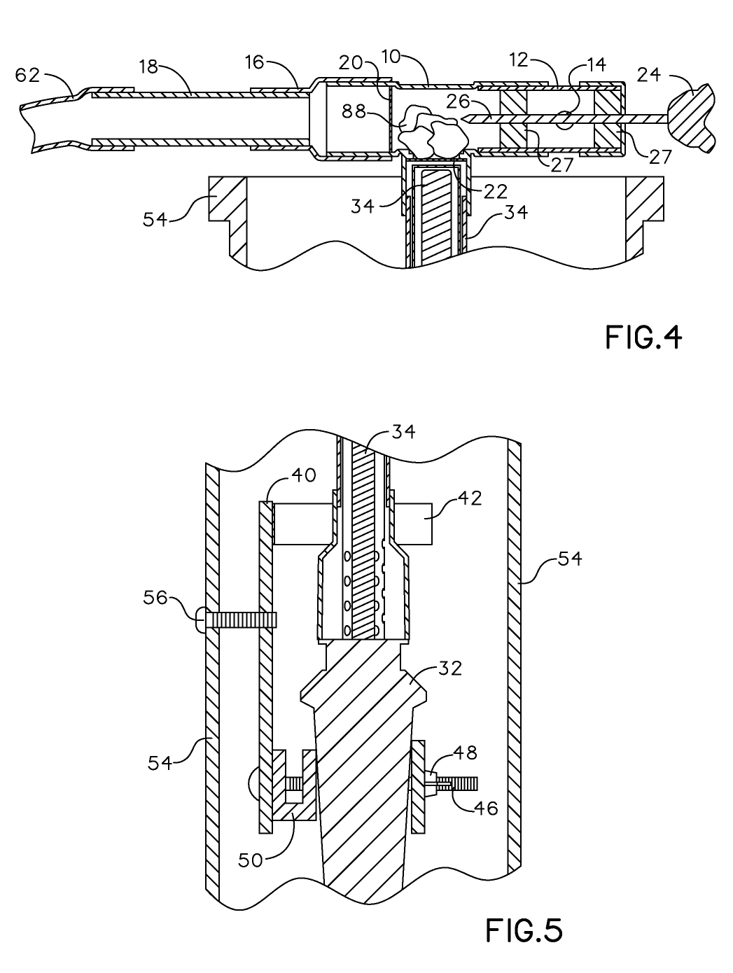

Referring now to FIGS. 1 through 8, the present invention may include an electric vaporizer 100. The electric vaporizer 100 may include a tubular base 84 for supporting an upper tube 54 in a vertical orientation, as illustrated in FIG. 3. The upper tube 54 may retain a heating assembly 31 and a screen assembly 21. The heating assembly 31 may be adapted for heating a medicament 88 so as to produce a vapor 90. The tubular base 84 may house a cooling assembly 71. The electric vaporizer 100 may be adapted so that the vapor 90 caused by the heated medicament 88 flows through a portion of the screen assembly 21 and then, via a flexible condenser tube 62, bypasses the upper tube before flowing through the cooling assembly 71 and to an outflow tube 64 for delivery to an end user.

The cooling assembly 71 may include a support sleeve 78 dimensioned and adapted to be properly supported by a bowl chamber 80, which in turn is supported by the tubular base 84. The bowl chamber 80 may form a cavity adapted to receive a cooling fluid 86, such as water, for cooling the vapor 90. The support sleeve 78 may form a sleeve aperture fluidly in communication with the cavity of the bowl chamber 80. A bowl plug 70 may be dimensioned and adapted to snugly fill the sleeve aperture so that the only fluid communication therethrough includes a plug condenser tube hole 72 and a plug outflow tube hole 74. The cooling assembly 71 may include a condenser elbow tube 66 and an outflow elbow tube 68 adapted to be snugly secured in the tube holes 72, 74, respectively, so that said only fluid communication is maintained through both. A bowl tube 76 may operatively engage the plug condenser tube hole 72 so as to protrude into the cavity and thus the cooling fluid of the bowl chamber 80, fluidly interconnecting the condenser elbow tube 66 thereto.

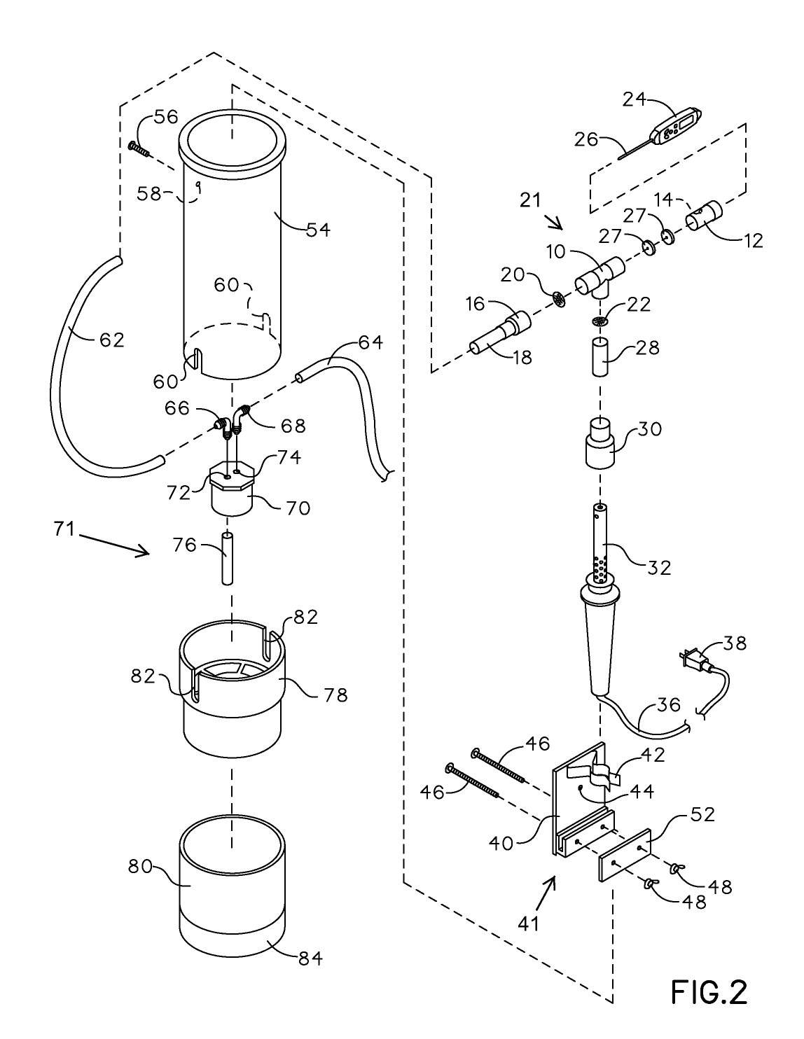

The support sleeve 78 may include a pair of bowl notches 82 formed along its periphery, as illustrated in FIG. 2. The lower portion of the upper tube 54 may form a pair of peripheral tube notches 60 dimensioned and adapted to align with the pair of the bowl notches 82 when the upper tube 54 and the support sleeve 78 are slidably engaged, as illustrated in FIGS. 2 and 3. When slidably engaged, each of the pair of aligned notches 60/82 may receive (in certain embodiments, sandwich) the flexible condenser tube 62 and the outflow tube 64 therein. Thereby allowing the tubing 62, 64 to controllably bypass the internal portions of the upper tube 54 which houses the heating element 34 and other components that may damage said tubing 62, 64 if too close. In certain embodiments, less than ten percent of the flexible condenser tube 62 protrudes in the upper tube 54 or the support sleeve 78.

The heating assembly 31 may be retained within the upper tube 54 by a retention assembly 41. The retention assembly 41 may include a soldering iron rear plate 40 providing a spring clip 42 and a spacer 50 on a first side, the spring clip 42 and the spacer 50 being spaced apart. The spring clip 42 and the spacer 50 may be separately adapted to removably secure two different portions along the heating assembly 31. The spacer 50 may include holder wing nuts 48 for securing a holder front plate 52 thereto. The retention assembly 41 may provide holder bolts 46 to connect to the upper tube 54. The retention assembly 41 may be connected to the upper tube 54 by a holder screw 56 interconnecting a tube screw hole 58 and a holder rear screw hole 44, as illustrated in FIGS. 2 and 5.

The heating assembly 31 may include a soldering iron 32 for housing a heating element 34. The heating element 34 may be adapted to provide a predetermined amount of heat to vaporize the medicament 88 housed in the screen assembly 21. The heating element 34 may be powered by a power source such as an iron cord 36 and an iron plug 38. A portion of the soldering iron 32 may be secured via soldering iron coupling 30 to the spring clip 42 of the retention assembly 41. The soldering iron 32 may be operably coupled to a vapor tee tube 10 of the screen assembly 21 via a tee extension 28, thereby allowing the predetermined amount of heat flow into the screen assembly 21 and the medicaments 88 housed therein.

The screen assembly 21 may provide a horizontal screen 22 and a vertical screen 20, both housed in the screen assembly 21. The horizontal screen 22 may be disposed between the heating element 34 and the medicament 88. In certain embodiments, the medicament 88 may be supported by the horizontal screen 22. The vertical screen 20 may be disposed between the medicament 88 and the flexible condenser tube 62.

The screen assembly 21 may include a vapor tee tube 10 for securing the vertical screen 20. The vapor tee tube 10 may provide a first egress and an opposing second egress, in addition to a thermal input coupled to the heating assembly 31 via the tee extension 28, wherein the vertical screen 20 is secured within the first egress, as illustrated in FIGS. 1-4. A condenser tube coupler 16 and a rigid condenser tube 18 may be adapted to interconnect the flexible condenser tube 62 and the vapor tee tube 10 via first egress. The vertical screen 20 may be adapted to filter the resulting vapor 90 flowing from the heated medicament 88.

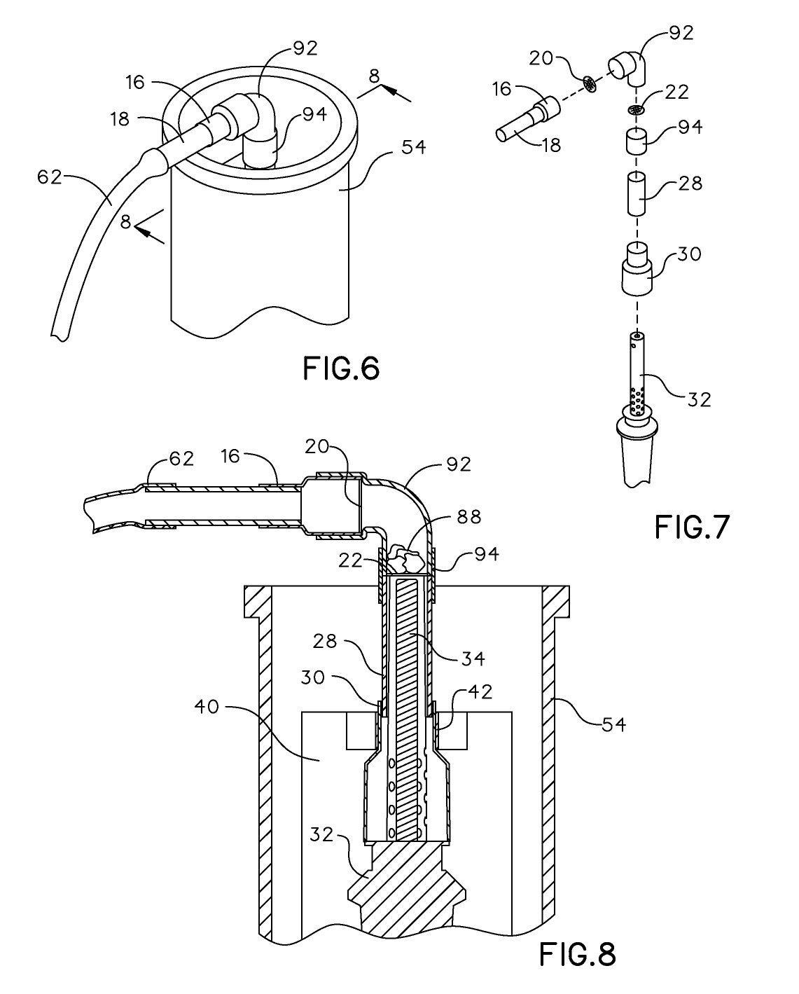

A thermal sensor 24 having a thermal sensor probe 26 may also be secured to the vapor tee tube 10, wherein a tee plug 12 and probe support plugs 27 operatively secure the thermal sensor probe 26 within a portion of the opposing (second) egress. A tee plug vent 14 may be formed within the tee plug 12. The thermal sensor 24 may be adapted to determine the temperature within the screen assembly 21. In certain embodiments, the vapor tee tube 10, the tee plug 12, the tee plug vent 14, and the condenser tube coupler 16 may be replaced by a vapor elbow tube 92 and an elbow coupling 94 having only one egress in addition to the thermal input coupled to the heating assembly 31, as illustrated in FIGS. 6-9.

A method of using the present invention may include the following. The electric vaporizer 100 described above may be provided. A user may place the medicament 88 on the horizontal screen 22 prior to attaching the screen assembly 21 to the heating assembly 31. The heating assembly 31, through the power source, urges the predetermined amount of heat over the medicament 88 so as to form the vapor 90. The user may utilize their lungs to provide air pressure to urge the vapor 90 through the screen assembly 21, through the flexible condenser tube 62, into the cooling assembly 71 via the condenser tube elbow 66 and to and through the outflow tube 64 and into said lungs, while using only one hand to hold the outflow tube 64.

It should be understood, of course, that the foregoing relates to exemplary embodiments of the invention and that modifications may be made without departing from the spirit and scope of the present invention.

What is claimed is:

1. An electric hookah, compromising:
a tubular base forming a cavity for receiving a cooling fluid;
an upper tube supported by the tubular base so that the only fluid communication with the cavity is via a condenser tube hole and an outflow tube hole;
a heating element housed in the upper tube;
a vapor tube having a thermal input and at least one egress, wherein the heating element is coupled to the thermal input;
a flexible condenser tube fluidly interconnecting a first egress of the at least one egress and the condenser tube hole; and
an outflow tube extending from the cavity and through the outflow tube hole.

2. The electric hookah of claim 1, further comprising a thermal sensor operatively coupled to a second egress of the at least one egress.

3. The electric hookah of claim 1, further comprising a support sleeve dispose between the tubular base and the upper tube, wherein an upper portion of the support sleeve slidably receives a lower portion of the upper tube.

4. The electric hookah of claim 3, wherein the support sleeve comprises a bowl plug forming the condenser tube hole and the outflow tube hole.

5. The electric hookah of claim 3, wherein the upper portion of the support sleeve forms a pair of the bowl notches, wherein the lower portion of the upper tube forms a pair tube notches dimensioned and adapted to align with the pair of the bowl notches when the upper tube is slidably received in the support sleeve, forming a pair of aligned notches.

6. The electric hookah of claim 5, wherein the pair of aligned notches receive a portion of the flexible condenser tube and the outflow tube, so that less than ten percent of the flexible condenser tube protrudes in the upper tube or the support sleeve.

7. The electric hookah of claim 1, further comprising two elbow tubes, a first elbow tube interconnecting the condenser tube hole and the condenser tube and a second elbow tube interconnecting the outflow tube hole and the outflow tube.

8. The electric hookah of claim 7, further comprising a bowl tube interconnecting the cavity and the first elbow tube.

9. The electric hookah of claim 8, further comprising a cooling fluid in the cavity so that a portion of the bowl tube is submerged therein.

10. An electric hookah, compromising:
a tubular base forming a cavity for receiving a cooling fluid;
an upper tube supported by the tubular base so that the only fluid communication with the cavity is via a condenser tube hole and an outflow tube hole;
a support sleeve dispose between the tubular base and the upper tube, wherein an upper portion of the support sleeve slidably receives a lower portion of the upper tube, wherein the support sleeve forms the condenser tube hole and the outflow tube hole, wherein the lower portion of the upper tube forms a pair tube notches;
a pair of aligned notches formed by the pairs of bowl and tube notches, wherein the pair of aligned notches receive a portion of the flexible condenser tube and the outflow tube, so that less than ten percent of the flexible condenser tube protrudes in the upper tube or the support sleeve;
a heating element housed in the upper tube;
a vapor tube having a thermal input and a first and a second egress, wherein the heating element is coupled to the thermal input;
a flexible condenser tube fluidly interconnecting the first egress and the condenser tube hole;
a thermal sensor operatively coupled to the second egress;
an outflow tube extending from the cavity and through the outflow tube hole;
two elbow tubes, a first elbow tube interconnecting the condenser tube hole and the condenser tube and a second elbow tube interconnecting the outflow tube hole and the outflow tube;
a bowl tube interconnecting the cavity and the first elbow tube; and
a cooling fluid in the cavity so that a portion of the bowl tube is submerged therein.
